(19) United States
(12) Patent Application Publication
(10) Pub. No.: US 2025/0387390 A1
(43) Pub. Date: Dec. 25, 2025

(54) METHODS FOR TREATING FIBROSIS USING PKM2 ACTIVATORS

(71) Applicant: Proda Biotech L.L.C., Marietta, GA (US)

(72) Inventors: Zhi-Ren LIU, Marietta, GA (US); Ganesh SATYANARAYANA, Marietta, GA (US)

(21) Appl. No.: 17/930,686

(22) Filed: Sep. 8, 2022

Related U.S. Application Data
(63) Continuation of application No. PCT/US2021/022009, filed on Mar. 11, 2021.
(60) Provisional application No. 62/987,946, filed on Mar. 11, 2020.

(51) Int. Cl. A61K 31/5025; A61K 31/095; A61K 31/194; A61K 31/34; A61K 31/496; A61K 31/519; A61K 31/661; A61K 31/7076; A61K 45/06; A61P 1/16; A61P 1/18; A61P 9/00; A61P 11/00

(52) U.S. Cl. CPC A61K 31/5025; A61K 31/095; A61K 31/194; A61K 31/34; A61K 31/496; A61K 31/519; A61K 31/661; A61K 31/7076; A61K 45/06; A61P 1/16; A61P 1/18; A61P 9/00; A61P 11/00

(57) ABSTRACT

A method of treating a patient suffering from disease that is caused by organ/tissue fibrosis includes the administration of a PKM2 activator. The PKM2 activator can reduce LOX protein expression and activate PKM2.

### TECHNICAL FIELD

This application relates to the prevention, treatment, and/or reversal of fibrosis. This application also relates to the use of activators of pyruvate kinase M2 (PKM2) for the treatment, prevention, or amelioration of fibrosis.

### BACKGROUND

Fibrosis is defined as abnormal accumulation of a fibrous tissue caused by e.g., tissue damage repair and/or immune reactions. In humans, fibrillation (fibrosis, fibrillation often referred to atrial fibrillation) is found in various organs and tissues such as lung, liver, pancreas, kidney, bone marrow and skin.

Collagen fibrils are excessively produced during fibrosis progression as a consequence of inflammation response to tissue damages. Fibrosis affects tissues of almost all organs. Myofibroblasts are the main cell type that is engaged in collagen synthesis and secretion. Collagen is an ECM protein that is mainly compose of Gly-x-Pro and Gly-x-Hyp triplet repeats. Glycine is the amino acid that makes up over 30% of the content of collagen. Myofibroblasts must engage in excessive glycine production in order to meet the needs of massive collagen production and secretion during fibrosis progression. Cells obtain glycine from two sources: (1) Glycine from food breakdown can be channeled to subsequent protein synthesis. (2) de novo biosynthesis.

Extracellular matrix (ECM) composition and stiffness are major driving forces for the development and persistence of fibrotic diseases. Lysyl oxidase (LOX) and LOX-like (LOXL) proteins play crucial roles in ECM remodeling due to their collagen crosslinking and intracellular functions.

Fibrogenesis is an essential process that is a critical part of tissue repair and wound healing. Excessive fibrosis is common in many rare and common disease conditions and is important in disease pathogenesis. Diseases characterized by excessive fibrosis include but are not restricted to: systemic sclerosis, scleroderma, hypertrophic cardiomyopathy, dilated cardiomyopathy (DCM), atrial fibrillation, ventricular fibrillation, myocarditis, liver cirrhosis, kidney diseases, diseases of the eye, asthma, cystic fibrosis, arthritis and idiopathic pulmonary fibrosis. Despite the large impact on human health, therapeutic and diagnostic approaches to fibrosis are still an unmet medical need.

### SUMMARY

One aspect of this application includes the treatment, prevention or alleviation of fibrosis in a subject in need of treatment through the administration of PKM2 activator(s). A method of treating a patient suffering from disease that is caused by organ/tissue fibrosis includes administering to the patient in need thereof a pharmaceutical composition comprising an effective amount of a PKM2 activator.

Another aspect includes a method in which the PKM2 activator is TEPP-46.

Another aspect includes a method of treating a patient suffering from disease that is caused by organ/tissue fibrosis and includes the administration of a PKM2 activator. The PKM2 activator can reduce collagen synthesis and LOX/L family expression in myofibroblasts. Treatment with PKM2 activator(s) can decrease myofibroblast apoptosis resistance.

### BRIEF DESCRIPTION OF THE DRAWINGS

FIG. 1 shows that TEPP-46 reverses liver and lung fibrosis and decreases serine/glycine metabolism in fibrotic liver and lung.

FIG. 2 shows that the dimer of PKM2 protects myofibroblasts from oxidative stress induced apoptosis by upregulation of NADPH metabolism.

FIG. 3 shows that PKM2 is expressed in myofibroblasts.

FIG. 4 shows treatment of fibroblasts with a PKM2 activator (DASA-10) decreases metabolic enzymes that are involved in serine/glycine metabolism.

FIG. 5 shows treatment with a PKM2 activator (TEPP-46) reverses pancreatitis.

FIG. 6 shows treatment with a PKM2 activator (TEPP-46) decreases cardiac fibrosis after cardiac infarction.

FIG. 7 shows treatment with a PKM2 activator (DASA-10) reduces LOX and LOXL2 expression in vivo in activated fibroblasts.

FIG. 8 shows treatment with a PKM2 activator (TEPP-46) reduces LOX expression in vivo in mouse models of liver and lung fibrosis.

FIG. 9 shows the effects of a PKM2 activator (Mitapivat) on collagen production in myofibroblasts.

### DEFINITIONS

By “administering” is meant a method of giving a dosage of a pharmaceutical composition to a patient. The compositions described herein can be administered by a route selected from, e.g., ocular, inhalation, parenteral, dermal, transdermal, buccal, rectal, vaginal, sublingual, perilingual, nasal, topical administration, and oral administration. Parenteral administration includes intravenous, intraperitoneal, subcutaneous, and intramuscular administration. The preferred method of administration can vary depending on various factors, e.g., the components of the composition being administered, and the severity of the condition being treated.

The term “fibrosis” refers to a condition involving the development of excessive fibrous connective tissue, e.g., scar tissue, in a tissue or organ. Such generation of scar tissue may occur in response to infection, inflammation, or injury of the organ due to a disease, trauma, chemical toxicity, surgery and so on. Fibrosis may develop in a variety of different tissues and organs, including the liver, kidney, intestine, lung, heart, etc. These changes are sometimes called fibrocystic changes and used to be called fibrocystic disease. The fibrosis, as used herein, is not cancerous fibrosis.

The term “inhibiting” or “inhibition,” as used herein, refers to any detectable positive effect on the development or progression of a disease or condition (e.g., fibrosis). Such a positive effect may include the delay or prevention of the onset of at least one symptom or sign of the disease or condition, alleviation or reversal of the symptom(s) or sign(s), and slowing or prevention of the further worsening of the symptom(s) or sign(s).

The term “effective amount” as used herein refers to an amount of compound (e.g., an activator of pyruvate kinase M2 (PKM2)) that produces an acute or chronic therapeutic effect upon appropriate dose administration. The effect includes the prevention, correction, inhibition, or reversal of the symptoms, signs and underlying pathology of a disease/condition (e.g., fibrosis of the liver, kidney, or intestine) and related complications to any detectable extent. The exact amount and dosing schedule will depend on the purpose of the treatment, and will be ascertainable by one skilled in the art using known techniques

The term “patient” means any animal, e.g., mammal or a human.

The term “Prodrug” is meant to indicate a compound that may be converted under physiological conditions or by solvolysis to a biologically active compound of the application. Thus, the term “prodrug” refers to a metabolic precursor of a compound of the application that is pharmaceutically acceptable. A prodrug may be inactive when administered to a subject in need thereof, but is converted in vivo to an active compound of the application. Prodrugs are typically rapidly transformed in vivo to yield the parent compound of the application, for example, by hydrolysis in blood. The prodrug compound often offers advantages of solubility, tissue compatibility or delayed release in a mammalian organism.

The terms “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The pharmaceutical composition can be administered parenterally, e.g., intravenously, subcutaneously, intradermally, or intramuscularly. Thus, the application provides compositions for parenteral administration that comprise a solution of the compound or salt dissolved or suspended in an acceptable carrier suitable for parenteral administration, including aqueous and non-aqueous isotonic sterile injection solutions.

The term “selective” means at least 20%, 50%, 75%, 2-fold, 3-fold, 4-fold, 5-fold, 6-fold, or 10-fold greater inhibition of a PKM2 over a second kinase, e.g., a second pyruvate kinase, e.g., a different isoform. Thus, in some embodiments, the agent is selective for PKM2 over another isoform. For example, an agent is selective for PKM2 relative to PKM1. Selective regulation, e.g., inhibition or activation, or selective modulation, are used interchangeably with specific regulation or specific modulation.

The term “therapy” includes not only prevention and treatment for fibroses but also maintenance for suppression of progression of fibrillation in tissues, mitigation of inflammation, mitigation of symptom associated with fibrosis and relapse prevention.

### DETAILED DESCRIPTION

The application features methods and kits that utilize activators of pyruvate kinase M2 (PKM2) for the treatment, prevention, or amelioration of diseases resulting from organ tissue fibrosis. This application provides methods of treating, ameliorating or preventing fibrosis or a fibrosis-associated disorder, as well as methods of screening for compounds and compositions useful in those methods. In certain examples, a method of treating a patient suffering from fibrosis including administering to the patient in need thereof a pharmaceutical composition (i.e., a PKM2 activator) comprising an effective amount of a compound selected from the compositions disclosed herein.

Examples of fibrosis include lung fibrosis, hepatic fibrosis, cardiac fibrosis, pancreatitis, renal fibrosis, prostatic hyperplasia caused by fibrillation, bone marrow fibrosis and scleroderma. In these fibroses, symptoms associated with fibrosis, such as inflammation and atrophy, are observed depending upon the organ with advanced fibrillation and the rate of progression. Thus, treatment for symptom associated with fibrosis is also included in the present application.

Other examples include Liver fibrosis; Hepatitis, cirrhosis, NASH (non-alcoholic steatohepatitis), ASH/AH, Primary biliary cholangitis, Primary sclerosing cholangitis; Lung fibrosis; Idiopathic pulmonary fibrosis, COPD, cystic fibrosis, scleroderma; Chronic kidney diseases; Diabetic nephropathy, cystic fibrosis, Glomerulonephritis/nephritis, Hypertensive nephrosclerosis, Allograft nephropathy; Pancreas fibrosis; Pancreatitis; Systemic sclerosis; Cardiac fibrosis; Cardiovascular fibrosis; Vascular fibrosis; coronary artery disease and peripheral artery disease, artery stiffness, Vasculitis; Primary myelofibrosis; Reducing scar in plastic surgery; Surgical complications due to fibrosis; and Radiation-induced fibrosis.

Myocardial infarction is an important complication of coronary artery disease and usually results from a critical reduction in coronary blood flow secondary to coronary thrombosis. The two important pathological changes of the cardiac tissue after acute myocardial infarction are fibrosis and hypertrophic growth of the cardiac tissues. Both changes (“remodeling”) significantly contribute to the pathogenesis of heart failure. Intravenous thrombolytic agent therapy has been widely used to restore flow to the occluded coronary artery. A thrombolytic agent is a medicament capable of lysing the fibrin-platelet thrombus, and thereby permitting blood to again flow through the affected blood vessel. Such agents include streptokinase, urokinase, prourokinase, reteplase, alteplase and tissue-type plasminogen activator (t-PA). The mortality of patients with acute myocardial infarction even if treated with thrombolytic agents remains high.

Patients treated using an above method may or may not have detectable fibrosis. In some embodiments, the patient has at least about a 5%, 10%, 20%, 30%, 40% or even 50% or more reduction in the amount of fibrosis present in the patient after administering PKM2 activators, after e.g. 1 day, 2 days, 1 week, 1 month or 6 months or more. Administering such a compound may be on, e.g., at least a daily basis. The delay of clinical manifestation of fibrosis in a patient as a consequence of administering a compound disclosed here may be at least e.g., 6 months, 1 year, 18 months or even 2 years or more as compared to a patient who is not administered a compound such as one disclosed herein.

In one example, PKM2 activators reduced expression of Lysyl oxidase (LOX), which is a member of the LOX/L family of proteins. This family of proteins is thought to promote covalent crosslinking of ECM proteins such as collagen and elastin via oxidation of lysyl amine residues, resulting in the establishment of the tensile strength of ECM. The LOX/L proteins family are expressed in various tissues and their dysregulation is associated with the pathology of all diseases of organ tissue fibrosis. The expression of LOX and the different LOX family, including LOXL2 and LOXL4 proteins varies in different diseases. This may be due to a number of reasons, such as the difference in tissue distribution, processing, domains, regulation of activity, as well as other differences between the proteins. Due to functional roles of LOX/L family proteins in progression of diseases of organ tissue fibrosis, inhibition of the LOX/L family has been an attractive strategy for developing treatment for fibrosis. Experiments have demonstrated that PKM2 activators strongly reduces expression and secretion of LOX/L family proteins in myofibroblasts and in fibrotic tissues and blood circulation of mouse models of liver and lung fibrosis. The functional role of PKM2 in reduction of LOX/L family suggest that PKM2 activators are excellent agents for fibrosis treatment.

Methods for administering the therapeutic agent (e.g., PKM2 activator) for fibrosis and the pharmaceutical composition for treating fibrosis of the present application are appropriately determined depending upon e.g., the dosage form; the age, sex and other conditions of the patient; and symptom of the patient. For example, tablets, pills, powders, granules, capsules, liquid preparations, suspensions and emulsions are orally administered. Injections are intravenously administered singly or in combination with a general complemental liquid such as glucose and amino acids, and further, if necessary, injections are intra-arterially, intramuscularly, intradermally, subcutaneously or intraperitoneally administered by themselves. Suppositories are intra-rectally administered. A PKM2 activator may be administered with other treatments or other PKM2 activators.

By “activator” is meant an agent that increases the level of pyruvate kinase activity of PKM2 from the state of inactive monomeric or dimeric form or maintains or increases the activity of active tetrameric form of PKM2 (e.g., in the presence of an endogenous inhibitor). Increasing activity can include reducing endogenous down-regulation of PKM2 by an endogenous inhibitor (e.g., an endogenous phosphotyrosine peptide or protein). The binding of phosphotyrosine-containing peptide with activated PKM2 results in dissociation of FBP and inactivation of PKM2. Autonomous growth signaling in proliferating cells or stimulation of fat cells by insulin leads to tyrosine phosphorylation cascades. An activator can exert its effect in a number of ways including one or more of the following: an activator can render PKM2 resistant to inhibition by an inhibitor, e.g., an endogenous inhibitor; an activator inhibits release of an activator, more specifically FBP; an activator can bind to PKM2 and prevent an endogenous inhibitor from promoting the release of an endogenous activator, more specifically FBP; or an activator can inhibit the dissolution or promote the reassembly of the subunits which make up PKM2, e.g., an activator can inhibit oxidation of sulfhydryl moieties on such subunits, e.g., inhibit the oxidation of cysteine residues. An activator of pyruvate kinase activity of PKM2 may be referred to as a “PKM2 activator.”

An activator can cause pyruvate kinase activity of PKM2 to increase to a level that is greater than PKM2's levels (e.g., basal levels) of activity (e.g., levels seen in the absence of an endogenous or natural activator/ligand, e.g., FBP). For example, the activator may mimic the effect caused by an endogenous or natural ligand or activator (e.g., FBP). The activating effect caused by the agent may be to the same, to a greater, or to a lesser extent than the activating effect caused by an endogenous or natural ligand or activator, but the same type of effect can be caused. Peptides, nucleic acids, and small molecules may be activators. In certain embodiments, the activator has a molecular weight in the range of 10 or 20, 100 or 200 to 10,000, 100 or 200 to 5,000, 100 or 200 to 2,000, or more preferably 100 to 300, 200 to 500, 150 to 500, 200 to 500, 300 to 500, or 150 to 800 Daltons or more.

Activators may be, e.g., peptides, nucleic acids, or small molecules. Peptides useful as activators in the methods, compositions, and kits described herein, can include modifications, e.g., in vivo or in vitro chemical derivatization of polypeptides (e.g., acetylation or carboxylation). Also included are modifications of glycosylation, e.g., those made by modifying the glycosylation patterns of a polypeptide during its synthesis and processing, or in further processing steps, e.g., by exposing the peptide to enzymes that affect glycosylation derived from cells that normally provide such processing, e.g., mammalian glycosylation enzymes. Also encompassed are versions of the same primary amino acid sequence that have phosphorylated amino acid residues, e.g., phosphotyrosine, phosphoserine, or phosphothreonine.

The application further relates to the compound or pharmaceutical composition as defined above, for use in combination with at least one therapeutically active agent selected from STAT inhibitors, other anti-inflammatory agents and/or immunosuppressant agents. The combination may be supplemented with one or more other active ingredients, e.g. anticoagulants, or surgical methods such as angioplasty.

Peptides useful as activators may be synthetic or purified from natural sources. The peptides may be available commercially or may be produced in recombinant or non-recombinant cells lines. Characterization of isolated peptidic activators may be accomplished using, e.g., solution assays, gel assays (e.g., SDS-PAGE), membrane-bound methods, antibodies, enzyme-linked immuno-sorbent assays (ELISA), or liquid-chromatography electron-spray ionization mass spectrometry (LCMS).

One exemplary activator of PKM2 is TEPP-46, which has the following formula:

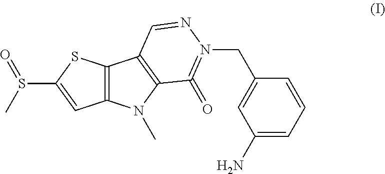

One exemplary activator of PKM2 is mitapivat, which has the following formula/structure:

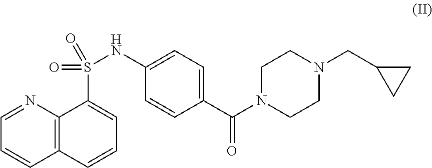 [SMILES: O=C(c1ccc(NS(=O)(=O)c2cccc3cccnc23)cc1)N1CCN(CC2CC2)CC1]

Other exemplary activators of PKM2 can be selected from the following candidates: 1,6-fructose-bis-phosphate, dithiothreitol, 2,5-anhydro-D-mannitol 1,6 bis-phosphate, AMP, phosphoenolpyruvate, and the following structures below:

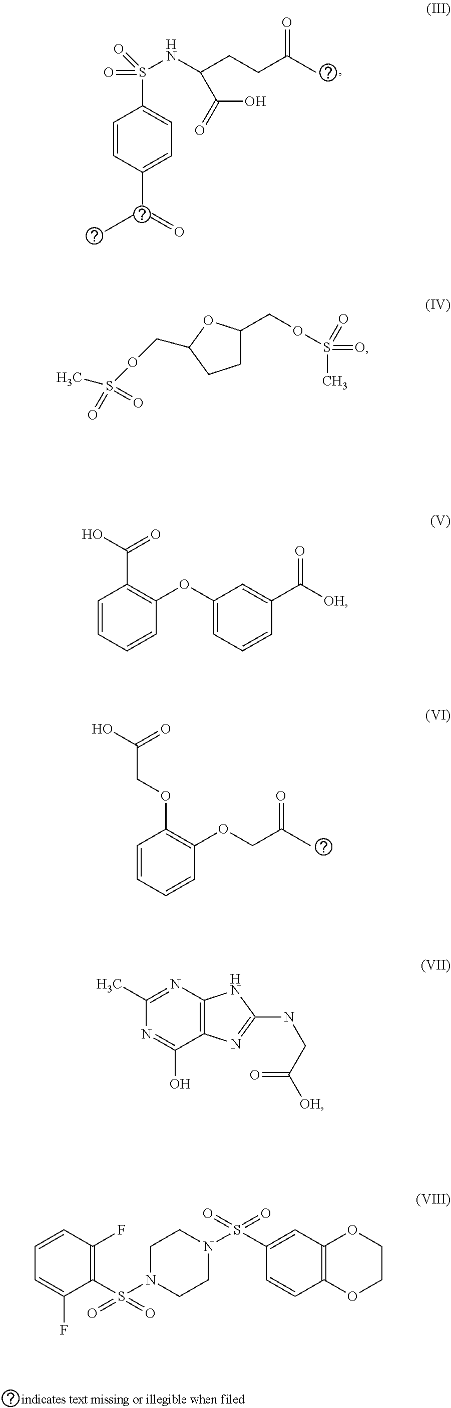

The pharmaceutical compositions of the application may be prepared by methodology well known in the pharmaceutical art. For example, a pharmaceutical composition intended to be administered by injection can be prepared by combining a compound of the application with sterile, distilled water so as to form a solution. A surfactant may be added to facilitate the formation of a homogeneous solution or suspension. Surfactants are compounds that non-covalently interact with the compound of the application so as to facilitate dissolution or homogeneous suspension of the compound in the aqueous delivery system.

The compounds (e.g., PKM2 activators) of the application, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

In cases of local administration or selective uptake, the effective local concentration of the drug may not be related to plasma concentration, and other procedures known in the art may be employed to determine the correct dosage amount and interval.

The amount of a PKM2 activators administered will, of course, be dependent on the subject being treated, the severity of the affliction, the manner of administration, the judgment of the prescribing physician, etc.

### EXAMPLES

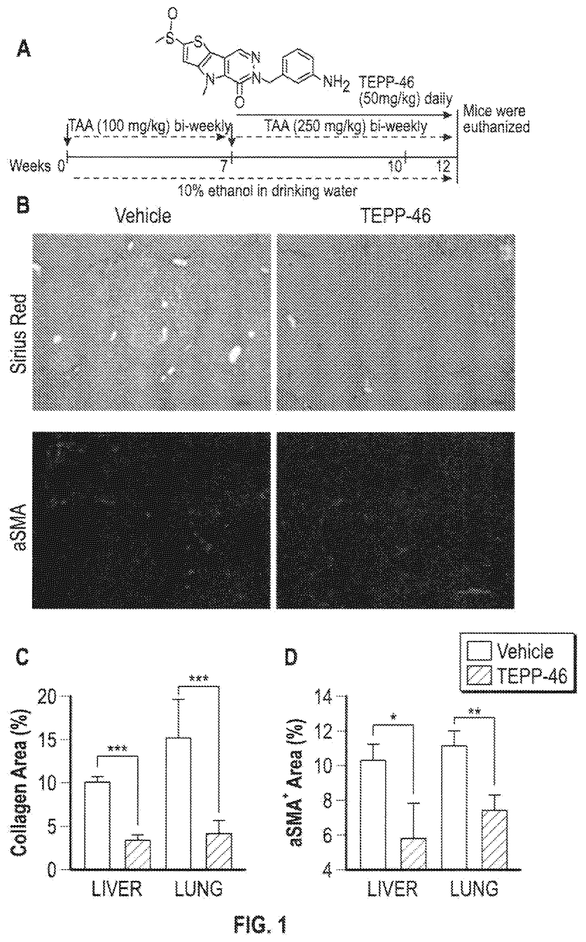

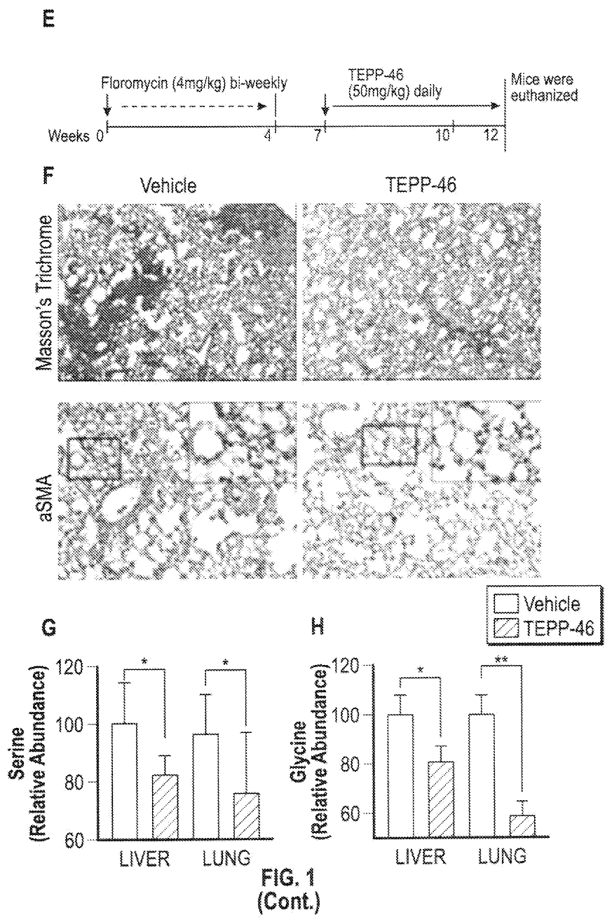

FIG. 1 shows that TEPP-46 reverses liver and lung fibrosis and decreases serine/glycine metabolism in fibrotic liver and lung. Sections (A) & (E) of FIG. 1 are schematic illustrations of the schedule of TAA/alcohol liver fibrosis (A) and bleomycin lung fibrosis (E) induction and subsequent TEPP-46 treatments. Section (B) of FIG. 1 shows representative images of Sirius red staining (upper) and a-SMA IF staining (bottom) of liver sections from mice treated with indicated agents. Sections (C) & (D) of FIG. 1 show quantitation of collagen levels in Sirius red (B) and Masson's trichrome (F) staining and a-SMA IF (B) and IHC (F) staining of fibrotic liver (B) and lung (F) using ImageJ software. Quantification was calculated from measurements of 10 mice. Four randomly selected tissue sections per animal and three randomly selected view fields in each section were quantified. The quantity of collagen levels in Sirius red or Masson's trichrome stain and α-SMA IF or IHC stain is presented as % of total area. Section (F) shows representative images of Masson's trichrome staining (upper) and α-SMA IHC staining (Bottom) of lung tissue sections from mice treated with indicated agents. Sections (G) & (H) show Quantitative analyses of Serine (G) and Glycine (H) levels in tissue extracts of liver and lung from mice treated with indicated agents by HPLC-ms. Serine and glycine levels are presented as relative abundance by defining the vehicle treated group as 100%. Error bars in Sections C, D, G, H represent mean±S.E.M

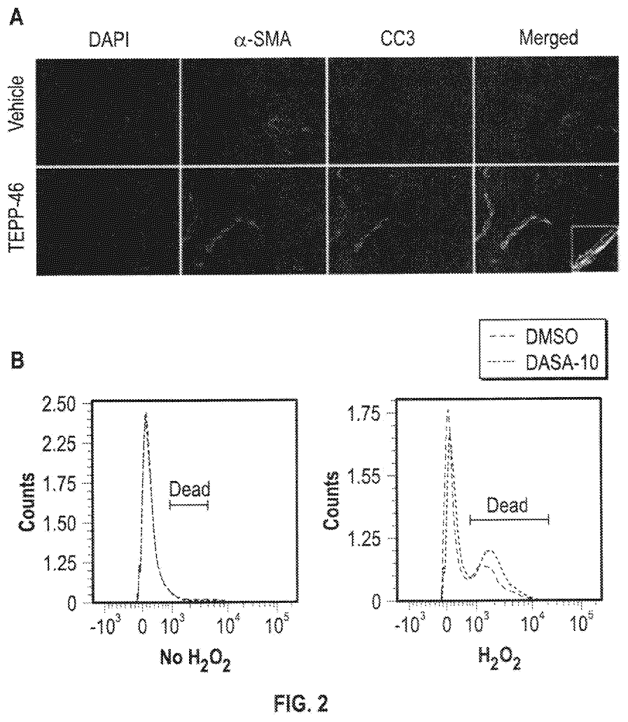

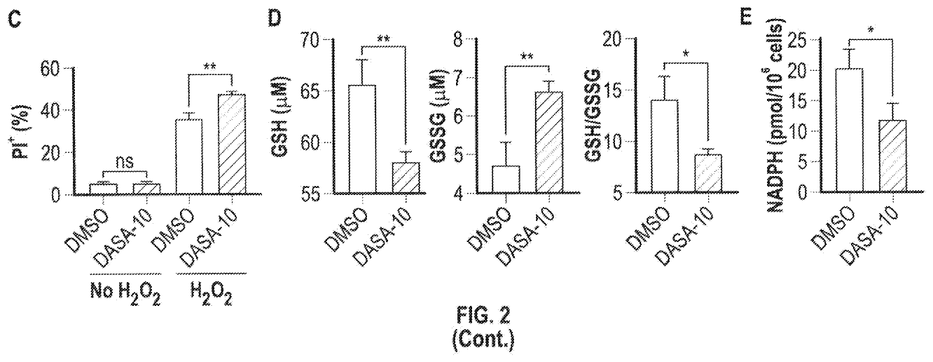

FIG. 2 shows that the dimer of PKM2 protects myofibroblasts from oxidative stress induced apoptosis by upregulation of NADPH metabolism. Representative images of IF co-staining of a-SMA with cleaved caspase 3 (CC3) of liver sections from mice treated with indicated agents are shown. Sections (B) & (C) Apoptosis of LX2 with (H₂O₂) or without (no H₂O₂) treatment and with DASA-10 or DMSO treatment was measured by FACS (graph, B) and (quantitation, C). Section (D) & (E) Cellular levels of GSH, GSSG, ratio GSH/GSSG (D), and NADPH (E) in LX2 were measured using kits. The cells were treated with either DMSA or DASA-10. GSH and GSSG are presented as mM (1×10⁶ cells lysate in 100 ml). NADPH is presented as pmole in 10⁶ cells. Error bars in C, D, E represent mean±S.E.M.

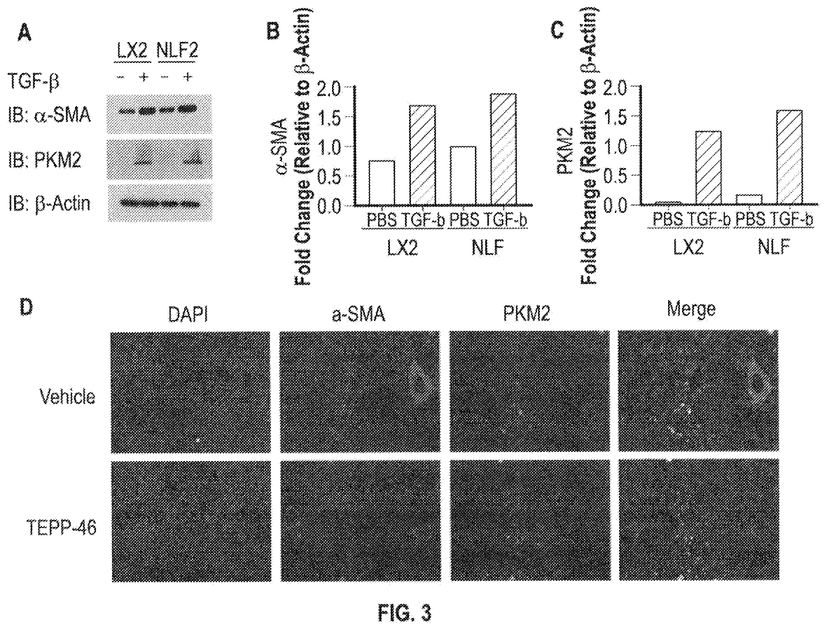

FIG. 3 shows a PKM2 is expressed in myofibroblasts. Section (A) shows levels of PKM2 (IB:PKM2) and α-SMA (IB:a-SMA) in LX-2 cells (LX2) and NLF with (+) or without (−) TGFβ treatment were analyzed by immunoblot. Immunoblot of b-actin (IB:b-actin) is a loading control. Sections (B) & (C) show quantification of a-SMA (B) and PKM2. Section (C) shows the levels in LX2 cells and NFL with (TGFb, black bar) or without (no TGFb, open bar) TGFb treatment. The PKM2 and a-SMA levels are presented as fold changes by comparison to controls. Error bars in Sections B, C, D, F represent mean±S.E.M. Section (D) shows representative images of IF co-staining a-SMA (red) with PKM2 (green) in liver sections from mice that were induced liver fibrosis by TAA-alcohol, and treated by TEPP-46 or vehicle. Co-staining (yellow) indicate PKM2 in myofibroblasts.

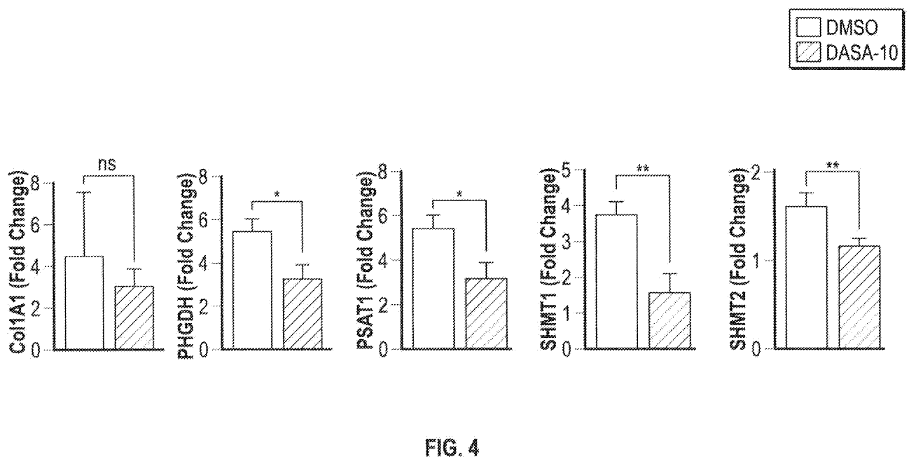

FIG. 4 shows a PKM2 activator (DASA-10) decreases metabolic enzymes that are involved in serine/glycine metabolism. Cellular levels of mRNA of collagen (Col1A1), PHGDH, PSAT1, SHMT1, and SHMT2 in LX2 cells with DASA-10 (black bar) or DMSO (open bar) treatment following TGFβ treatment were analyzed by qRT-PCR. The cellular mRNA levels are presented as fold change by comparing controls. Error bars represent mean±S.E.M.

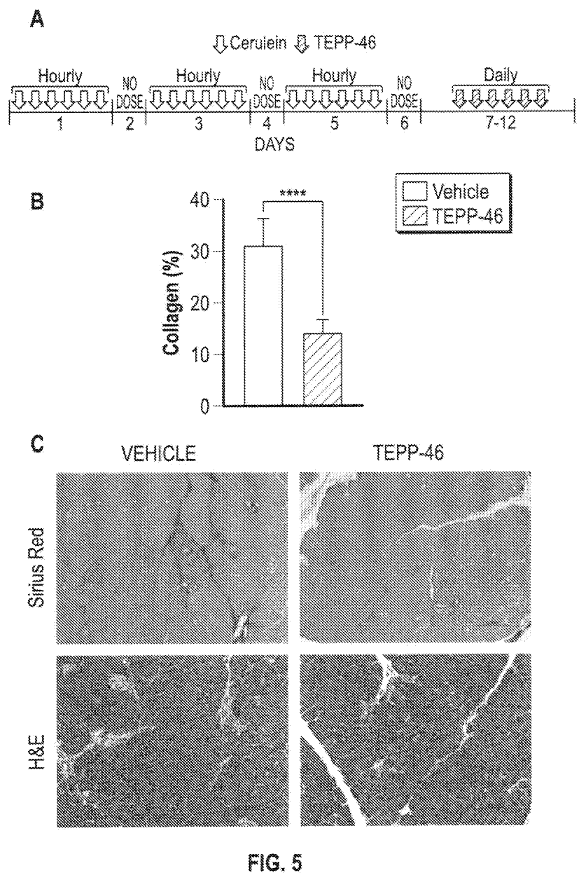

FIG. 5 shows that a PKM2 activator reverse pancreatitis. Section (A) is schematic illustration of the schedule of cerulein (red arrows) pancreatitis induction and subsequent TEPP-46 treatments (green arrows). Section (B) shows quantitation of collagen levels in Sirius red staining in (C) using ImageJ software. Quantification was calculated from measurements of 6 mice. Four randomly selected tissue sections per animal and three randomly selected view fields in each section were quantified. The quantity of collagen levels in Sirius red stain is presented as % of total area. Error bars in B represent mean±S.E.M. Section (C) shows representative images of Sirius red staining of collagen (Upper) and H&E staining of sections of pancreas from both vehicle and TEPP-46 treated animals.

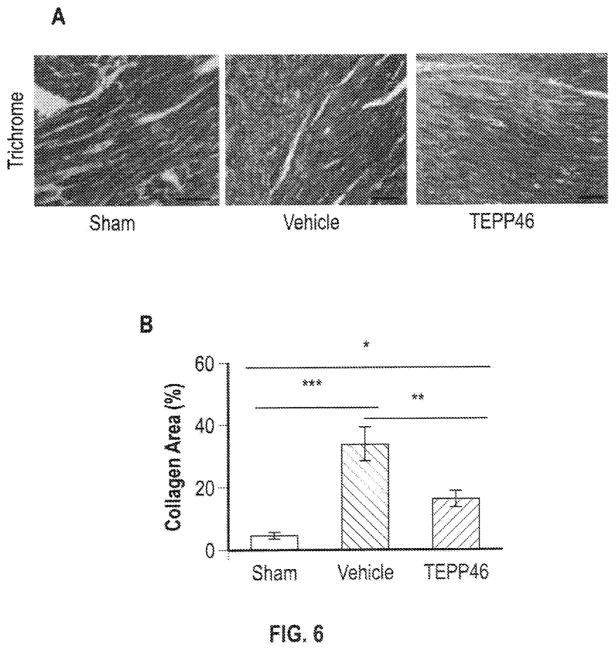

FIG. 6 shows that treatment of a PKM2 activator decreases cardiac fibrosis after infarction. C57BL/6J mice (n=6/group) were induced myocardial infarction (MI) by left ventricle ligation (LV). Sham are the mice underwent the same surgical procedure without LV. After 70 minutes, the LV was relieved. All mice were immediately treated with TEPP-46 or vehicle by i.p. injection. No treatment for the Sham mice. Second (A) shows represent images of Masson's trichrome staining of tissue sections from infarct areas of mice treated with indicated agent. Blue shows collagen staining. Scale bars, 100 mm. Section (B) shows quantification of trichrome staining of collagen in tissue sections. The quantification is a mean value of randomly selected 4 fields from each section. Four sections were randomly selected from each mouse. The quantification is presented as blue staining Collagen Area (%). Error bars represent mean±S.E.M. * P<0.05, ** P<0.01, *** P<0.001, by unpaired two-tailed Student's t-test.

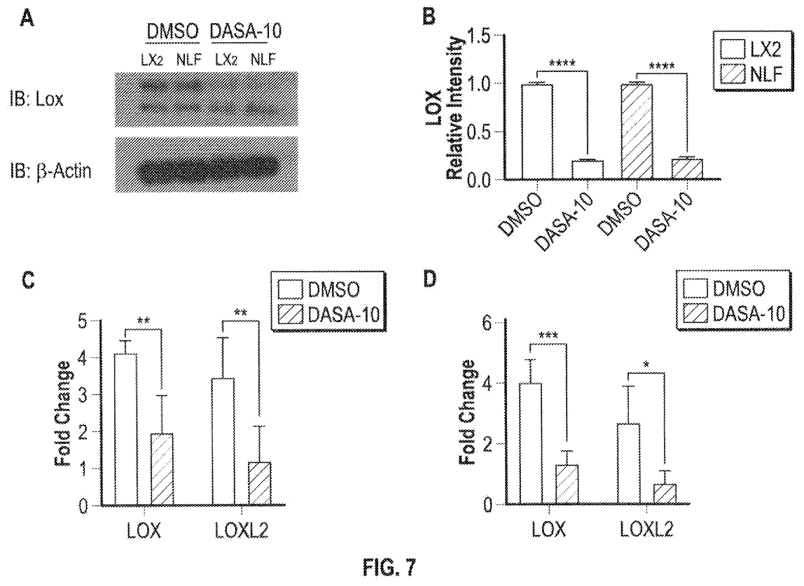

FIG. 7 shows DASA-10 reduces LOX and LOXL2 expression. Section (A) shows 1×10⁶ LX2 and NLF cells were treated with either DMSO or 10 μM DASA-10 and cultured under hypoxia for 24 hours. Section (B) Relative band intensities were calculated for both LOX and LOXL2 using ImageJ. Section (C) shows mRNA isolated and converted into cDNA and qPCR was performed for the expression of LOX and LOXL2 in LX2 cells. Section (D) shows mRNA isolated and converted into cDNA and qPCR was performed for the expression of LOX and LOXL2 in NLF cells.

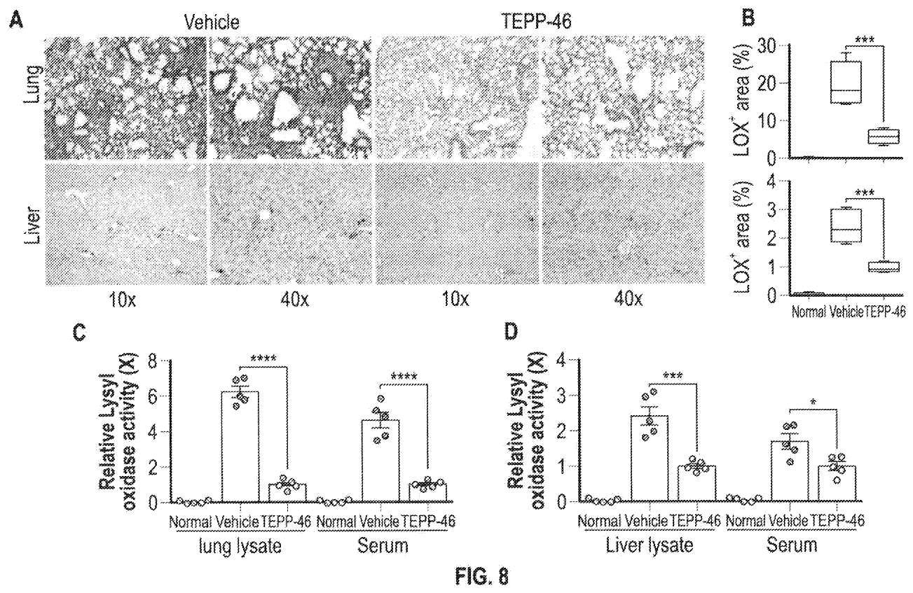

FIG. 8 shows TEPP-46 reduces LOX expression in vivo. Section (A) shows Fibrotic lungs (top panel) and liver (bottom panel) tissues from mice treated with indicated agents were sectioned at 5 μm and probed for LOX via immunohistochemistry staining. Section (B) shows LOX positive area was quantified using ImageJ in both lungs (top panel) and liver (bottom panel). Section (C) shows LOX activity assay was measured using a LOX activity kit in lung lysates (left columns) and serum (right columns) in bleomycin model of lung fibrosis. Section (D) shows LOX activity assay was measured using a LOX activity kit in liver lysates (left columns) and serum (right columns) in TAA/alcohol model of liver fibrosis.

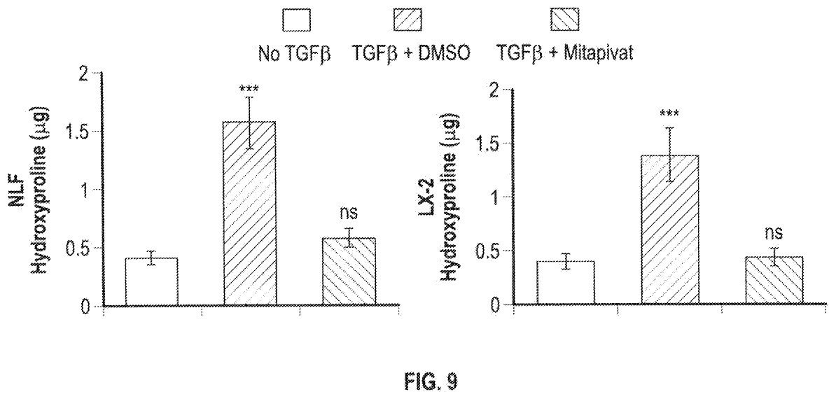

FIG. 9 shows the effects of a PKM2 activator (Mitapivat) on collagen production in myofibroblasts. Sections (A) & (B) include hydroxyproline in LX2 in which section (A) and lung fibroblasts (NFL) and section (B) with (filled bars) or without (open bars) TGFβ treatment was analyzed using a hydroxyproline kit. The cells were treated with Mitapivat (grey bars) or vehicle (black bars) after TGFβ treatment. The hydroxyproline in A and B is presented as μg of hydroxyproline in lysate of 1×10⁶ cells.

1. A method of treating a patient suffering from disease that is caused by organ/tissue fibrosis, the method comprising administering to the patient in need thereof a pharmaceutical composition comprising an effective amount of a PKM2 activator, wherein the the PKM2 activator reduces LOX protein expression and activates PKM2.

2. The method of claim 1, wherein the fibrosis is of the lung.

3. The method of claim 1, wherein the fibrosis is of the pancreas or pancreatitis.

4. The method of claim 1, wherein the PKM2 activator is TEPP-46

5. The method of claim 1, wherein the fibrosis is of the liver, kidney, bone marrow, heart, pancreas, skin, intestine, vasculature, or joints.

6. The method of claim 1, wherein the fibrosis is cholangitis or alcoholic hepatitis.

7. The method of claim 1, wherein the fibrosis is nonalcoholic steatohepatitis (NASH).

8. The method of claim 1, wherein the fibrosis is of the lung, including IPF and COPD.

9. The method of claim 1, wherein the fibrosis is idiopathic pulmonary fibrosis.

10. The method of claim 1, wherein the fibrosis is of the heart after cardiac surgery from a heart attack.

11. The method of claim 1, wherein the fibrosis is in the surgery complications and scar.

12. The method of claim 1, wherein the PKM2 activator has the following structure:

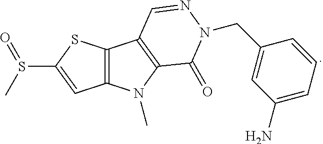 [SMILES: Cn1c2cc(S(C)=O)sc2c2cnn(Cc3cccc(N)c3)c(=O)c21]

13. The method of claim 1, wherein the PKM2 activator has the following structure:

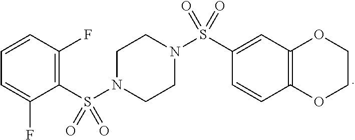 [SMILES: O=S(=O)(c1ccc2c(c1)OCCO2)N1CCN(S(=O)(=O)c2c(F)cccc2F)CC1]

14. The method of claim 1, wherein the PKM2 activator has the following structure:

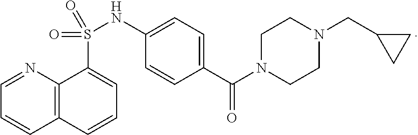 [SMILES: O=C(c1ccc(NS(=O)(=O)c2cccc3cccnc23)cc1)N1CCN(CC2CC2)CC1]

15. The method of claim 1, wherein the PKM2 activator has the following structure:

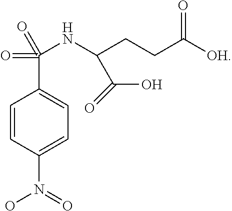

16. The method of claim 1, wherein the PKM2 activator has the following structure:

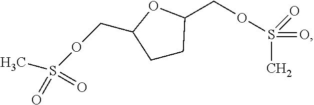 [SMILES: CS(=O)(=O)OCC1CCC(COS(C)(=O)=O)O1]

17. The method of claim 1, wherein the PKM2 activator has the following structure:

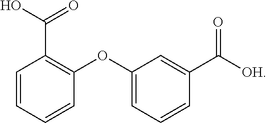 [SMILES: O=C(O)c1cccc(Oc2ccccc2C(=O)O)c1]

18. The method of claim 1, wherein the PKM2 activator has the following structure:

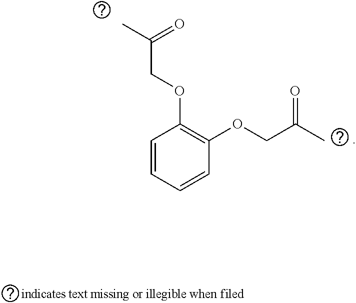 [SMILES: CC(=O)COc1ccccc1OCC(C)=O]

19. The method of claim 1, wherein the PKM2 activator has the following structure:

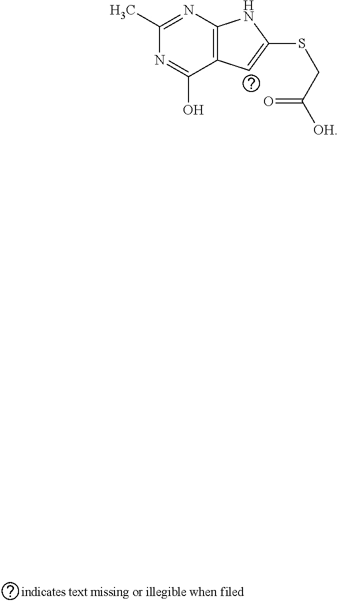 [SMILES: Cc1nc(O)c2cc(SCC(=O)O)[nH]c2n1]

20. A method of activating PKM2 in a mammal to decrease organ tissue fibrils synthesis and LOX protein expression in the subject and increase in myofibroblast apoptosis in need thereof, the method comprising administering to the mammal an effective amount of a PKM2 activating compound, to the subject.

21. The method of claim 20, wherein the PKM2 activator includes a pharmaceutically acceptable carrier, diluent or excipient.

22. The method of claim 20, further comprising administering an effective amount of one or more chemotherapeutic agents to the subject.

23. The method of claim 20, wherein the therapeutically effective amount of the compound is about 0.5 mg to about 1000 mg for human.

24. A method of treating a fibrotic disease in a person in need thereof, the method comprising administering to the person an effective amount of a PKM2 activator, wherein the PKM2 activator is TEPP-46.

25. The method of claim 24, wherein the fibrotic disease is liver fibrosis, lung fibrosis, heart fibrosis, skin fibrosis, bone marrow fibrosis, or intestinal fibrosis.

26. The method of claim 25 wherein the PKM2 activator is administered as a pharmaceutical composition comprising an excipient, dispersant, solubiliser, stabilizer and/or preservative.

27. The method of claim 25 wherein the PKM2 activator is administered orally or parenterally.

28. The method of claim 25, wherein the PKM2 activator is 1,6-fructose-bis-phosphate, dithiothreitol, 2,5-anhydro-D-mannitol 1,6 bis-phosphate, AMP, or phosphoenolpyruvate.

29. The method of claim 25, wherein the PKM2 activator reduces the express of the LOX/L family members.

30. A method of inhibiting fibrosis in a patient in need thereof or at risk thereof, said method comprising administering a therapeutically effective amount of a PKM2 activator to said patient wherein said fibrosis is associated with surgery.
